(19) United States
(12) Patent Application Publication
(10) Pub. No.: US 2014/0088348 A1
(43) Pub. Date: Mar. 27, 2014

(54) Rectal Prolapse Treatment Methods, Devices, and Systems

(71) Applicant: AMS Research Corporation, Minnetonka, MN (US)

(72) Inventors: Janet L. DeMarchi, Minnetonka, MN (US); Amanda Heys, Eden Prairie, MN (US)

(21) Appl. No.: 14/025,748

(22) Filed: Sep. 12, 2013

Related U.S. Application Data
(60) Provisional application No. 61/700,160, filed on Sep. 12, 2012.

(51) Int. Cl. A61F 2/00

(52) U.S. Cl. CPC A61F 2/0045
USPC 600/37

(57) ABSTRACT

Various surgical systems, devices and methods are provided for treating rectal prolapse, including a transobturator posterior rectal suspension method, a transobturator bilateral mesh arm rectal suspension method, a perineal approach rectal suspension method, a single incision perineal approach rectal suspension method, a single incision posterior rectal suspension method, and a perineal single incision rectopexy suspension method.

### PRIORITY

This application claims priority to and the benefit of U.S. Provisional Patent Application No. 61/700,160, filed Sep. 12, 2012, which is incorporated herein by reference in its entirety.

### FIELD OF THE INVENTION

The present invention relates generally to surgical methods and apparatus and, more specifically, to surgical devices, methods and systems for treatment of rectal prolapse.

### BACKGROUND OF THE INVENTION

Pelvic health for men and women is a medical area of increasing importance, at least in part due to an aging population. Examples of common pelvic ailments include incontinence (e.g., fecal and urinary), pelvic tissue prolapse (e.g., rectal prolapsed and female vaginal prolapse), and conditions of the pelvic floor.

Anal incontinence is a common problem that occurs in both men and women, though is certainly more prevalent in women after vaginal childbirth, presumably the result of trauma to pelvic floor muscles, supporting fascia and nerves. Fecal incontinence affects an estimated 7.6 percent of women between the ages of 30-90. The prevalence increases with age. Several factors contribute to anal continence, including the resting tone of the external and internal anal sphincters, as well as the position of the levator ani muscles, especially the puborectalis muscle, which forms a sling around the rectum and is responsible for the so-called “ano-rectal angle,” which keeps stool in the rectum until voluntary defecation relaxes the puborectalis muscle and straightens the angle, allowing stool to move towards the anus.

Defecation is often aided by expulsive abdominal forces. Anal incontinence may occur as the result of several mechanisms, including direct damage to the internal or external anal sphincters (from iatrogenic episiotomy or spontaneous lacerations during vaginal delivery), or to the levator ani muscles. It may also result from indirect injury of these muscles through denervation of the nerves that supply these muscles. Treatment of this problem has centered on pelvic floor rehabilitation, dietary changes, or surgical correction. Surgery has been used to treat specific defects in the anal sphincters, such as external anal sphincteroplasty. Success rates of only 50% or less are generally reported for these procedures on long-term follow-up.

Rectal prolapse typically occurs over time from straining to evacuate stool. As a person strains the connective tissue holding the rectum in place becomes weakened and the rectum can protrude from the anus. Other contributing factors are a weak anal sphincter and weak pelvic floor. Women are six times more likely to develop rectal prolapsed than men. Risk factors for a weakened pelvic floor are vaginal childbirth, aging and the effect of hormonal changes in menopause. The rectal prolapse may contribute to fecal incontinence and be the result of chronic constipation.

There remains a need to provide improved treatment devices, methods and systems, including techniques and tools that would address rectal suspension in a minimally invasive fashion.

### SUMMARY OF THE INVENTION

The present invention is directed to systems, devices, and methods for treating rectal prolapse, including a transobturator posterior rectal suspension method, a transobturator bilateral mesh arm rectal suspension method, a perineal approach rectal suspension method, a single incision perineal approach rectal suspension method, a single incision posterior rectal suspension method, and a perineal single incision rectopexy suspension method. Each implant method of the present invention addresses rectal suspension in a minimally invasive manner utilizing various implant systems and devices.

Further details of the aspects and advantages of each of the above are provided in this specification and in the appendix hereto, which is incorporated herein by reference in its entirety. Each of the above techniques and aspects are directed to minimally invasive approaches to rectal prolapsed treatment.

Additional aspects, features and advantages of the present invention will be apparent from review of the entirety of this application. The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is an illustration of various relevant anatomical features of the human pelvis region.

FIGS. 2-4 depict various implant devices having a support portion and extending arms, in accordance with embodiments of the present invention.

FIG. 5 depicts a helical needle device and connectivity to an implant portion, in accordance with embodiments of the present invention.

FIGS. 6-8 depict surgical methods for deploying an implant via a Kraske incision or procedure, in accordance with embodiments of the present invention.

FIG. 9 illustrates relevant anatomical structure and incisions for an implantation procedure, in accordance with embodiments of the present invention.

FIGS. 10-11 depict arms of an implant system, in accordance with embodiments of the present invention.

FIG. 12 illustrates relevant anatomical structure and a perineal incision for an implantation procedure, in accordance with embodiments of the present invention.

FIG. 13 depicts a generally Y-shaped implant, in accordance with embodiments of the present invention.

FIG. 14 depicts a long curved needle introducer device, in accordance with embodiments of the present invention.

FIGS. 15-16 depict implants having separate support portions and anchor arms, in accordance with embodiments of the present invention.

FIG. 17 depicts a curved needle introducer device, in accordance with embodiments of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF PREFERRED EMBODIMENTS

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings, unless specifically claimed, are illustrated only for ease of understanding, but not to limit the actual scale and dimension.

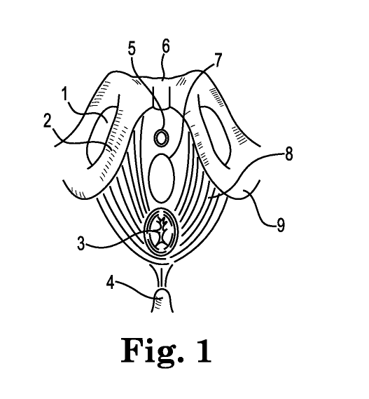

FIG. 1 shows the anatomy of the pelvis, including the pubic symphysis 6, the ischiopubic ramus 2, the ischial tuberosity 9, the coccyx 4, the obturator foramen 1, and the relationship of the levator ani muscles (and, in particular, the puborectalis 8) to the urethra 5, vagina 7, and the rectum 3.

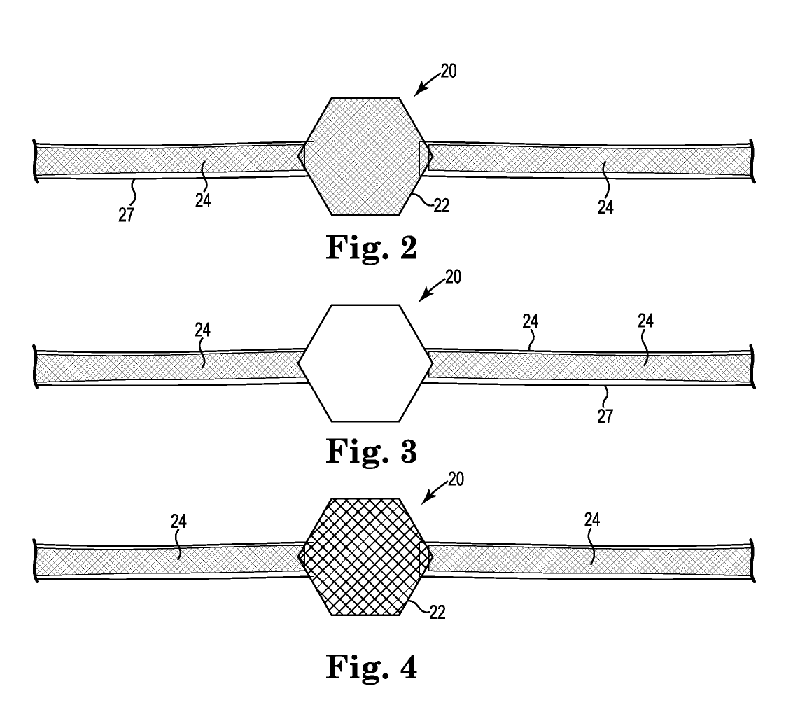

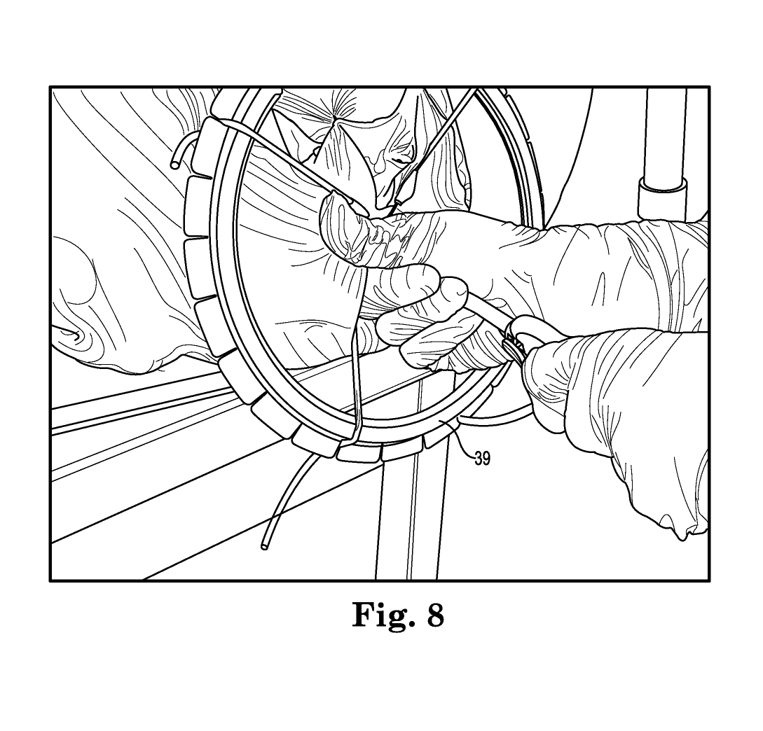

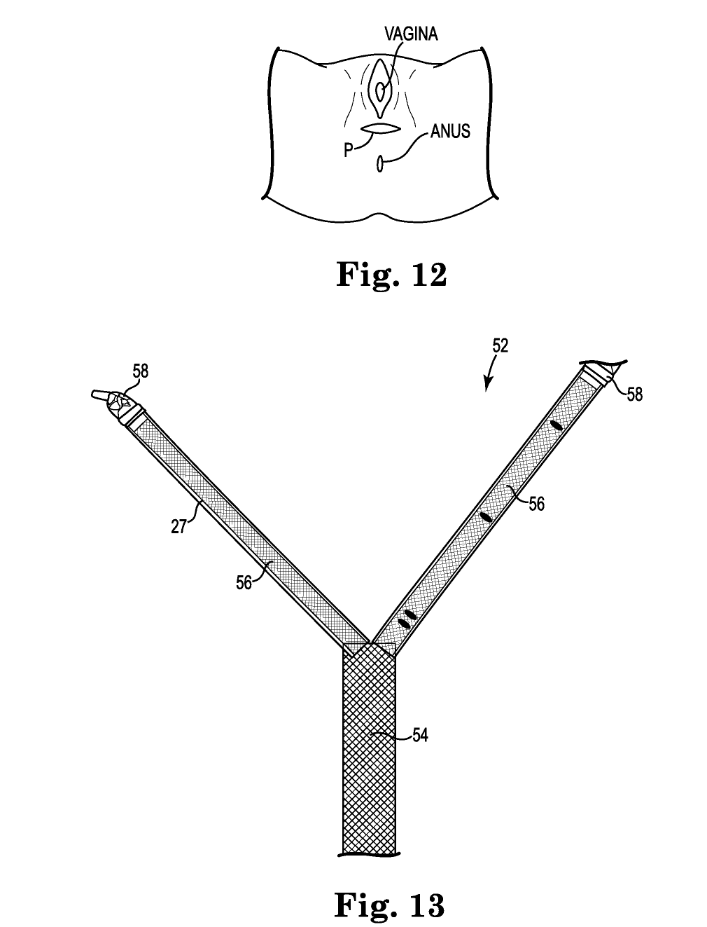

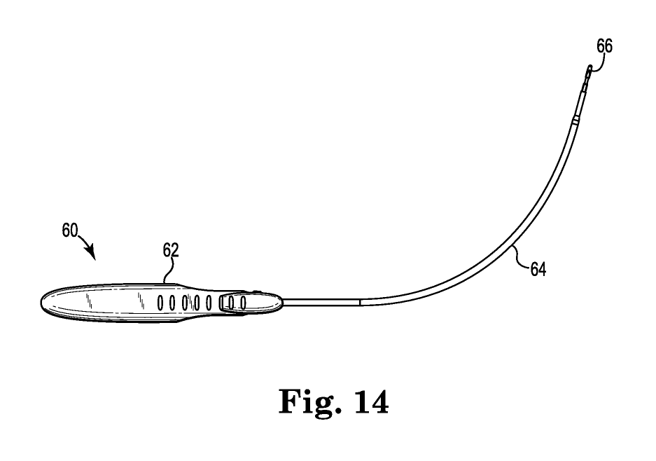

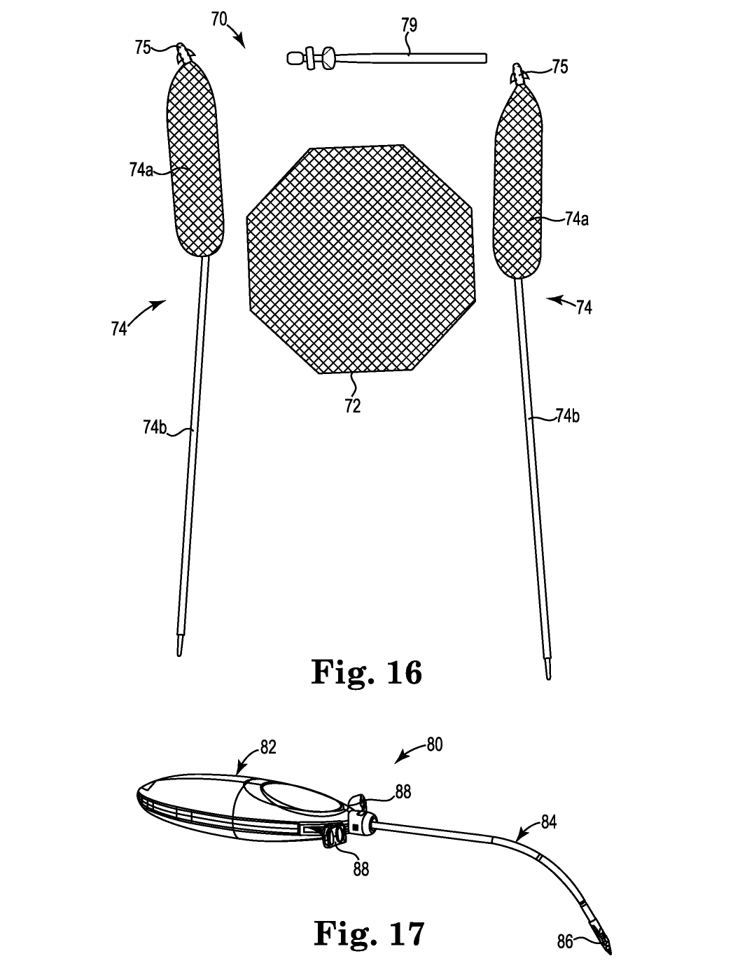

Referring generally to FIGS. 2-17, a plurality of systems, devices, and methods for treating rectal prolapse are provided, including a transobturator posterior rectal suspension method, a transobturator bilateral mesh arm rectal suspension method, a perineal approach rectal suspension method, a single incision perineal approach rectal suspension method, a single incision posterior rectal suspension method, and a perineal single incision rectopexy suspension method.

### Transobturator Posterior Rectal Suspension

Referring to FIGS. 2-8, embodiments of the present invention can include a system and method of deploying an implant or sling under the rectum to treat rectal prolapse. An implant 20 for use with such a system can include a body support portion 22 and extending arms 24. A distal portion of the arms 26 can include an anchor, dilator, needle or like member or device adapted to engage with an introducer needle. The support portion 22 can take on a wide variety of shapes and sizes. In certain embodiments, the support portion 22 is generally rectangular, square, oval, hexagonal (e.g., FIGS. 2-4), and the like. The support portion 22 and the arms 24 can be constructed of a polymer or like mesh material. In various embodiments, the support portion 22 can be constructed of a generally flexibly non-mesh material, as shown in FIG. 3. In certain embodiments, the support portion 22 can be approximately 6 cm×6 cm in size or profile.

A wide body of support is provided under the rectum with the employment of implant 20. To facilitate accurate placement of the support portion 22 under, and flat relative to the rectum, a Kraske incision K can be used for introduction and deployment of the implant 20. A Kraske incision is a transverse incision approximately 2 cm below the anus.

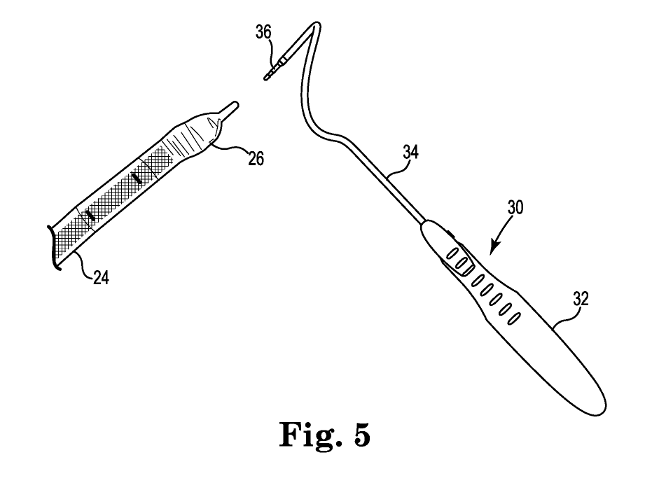

The implant system 20 can include an transobturator needle device 30. The needle device 30 can include a handle 32 and a needle 34, with the needle further including a distal end or tip 36 adapted to engage or secure with the end 26 of the arms 24. In certain embodiments, such as that shown in FIG. 5, the needle 34 includes a helical portion. The needle 34 can also be straight or curved in other embodiments. Helical embodiments can be advantageous because surgeons may have their fingers directly under the descending ramus during deployments and such a helical shape will facilitate travel to that point of contact.

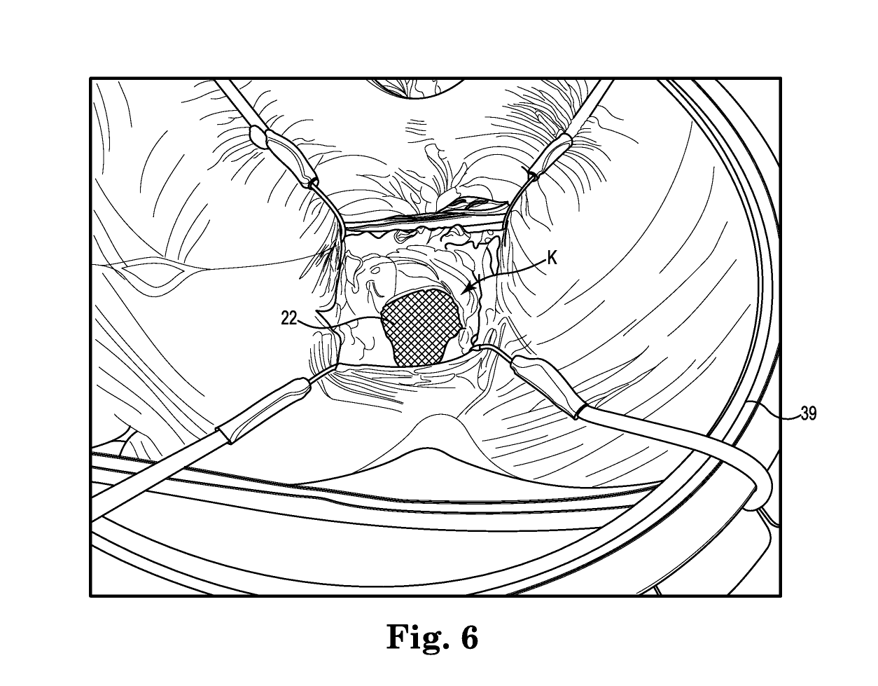

As shown in FIGS. 6-8, the patient can be placed in the lithotomy position and an approximately 3 cm Kraske incision can be performed or created (e.g., approximately 2 cm inferior to the anus). A Lone Star or like retractor 39 can be used to improve visualization and facilitate introduction of the implant 20. A blunt dissection is made to approximately 1-2 cm beyond the anorectal junction (e.g., transition from the rectum to the anal canal), and the wound is irrigated. The surgeon can then mark thigh incisions for passage of the transobturator needle device 30, on the most medial edge of the inferior pubic ramus—approximately at the level of the urethra. Next, the helical needle is passed through the medial thigh incision and lateral to the levator muscle. The needle will generally pass approximately 2-3 cm before meeting the surgeon's finger in the main incision, lateral to the rectum. One of the arms 24 are then attached to the distal end 36 of the needle 30 and pulled through the obturator foramen. Having the arms 24 anchored in the obturator foramen provides a beneficial anchor point and sufficient lift to the rectum. The same steps are repeated on the other side, using the opposing arm 24 of the implant 20. The surgeon can then pull up on the arms 24 to tension the portion 22 so that it lays generally flat against the rectum. For those embodiments including arm sheaths 27, the sheaths can then be removed to secure the mesh structures in place. Additionally, tacking sutures can be placed at edges or corners of the support portion 22 for securement with the rectum. At this point the arms 24 are extending through the thigh incisions and can be cut off accordingly at the level of the thigh incisions. The post anal incision can then be irrigated and closed in several layers.

### Transobturator Bilateral Mesh Arms Rectal Suspension

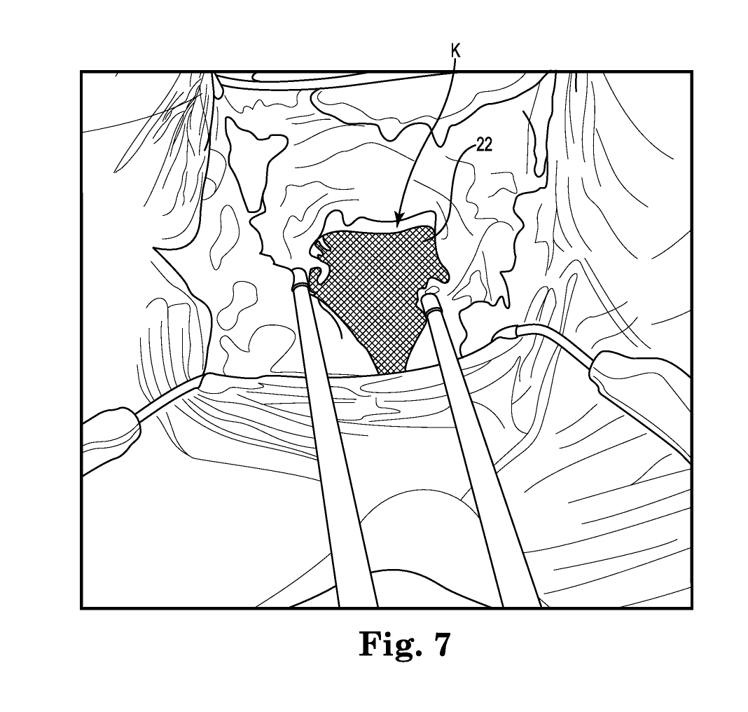

Referring to FIGS. 9-11, embodiments of the present invention can include a transobturator bilateral mesh arm implant system 40 and method to treat rectal prolapse. The implant system can include two mesh arms 42. Each arm 42 can be generally T-shaped and can include a base portion 44, and an extension portion 46, with the extension portion 46 including a distal tip or end portion 48. The end portions 48 can include an anchor, dilator, needle or like member or device adapted to engage with an introducer needle. The base portions 44 can take on wide variety of shapes and sizes. The base portions 44 and extension portions 46 can be constructed of a polymer or like mesh material.

The implant system 40 uses bilateral perianal incisions wherein the base portions 44 are sutured or otherwise engaged with the lateral walls of the rectum and the extension portions 46 are passed through the obturator foramen to anchor and lift the rectum. As with other embodiments disclosed herein, a helical needle device 30 can be employed to introduce and deploy the system 40. Other shaped needles can also be used without deviating from the scope of the present invention.

First, the patient is placed in the lithotomy position, and two bilateral vertical perianal incisions VP are created (FIG. 9). A blunt dissection is made approximately 5 cm along the rectum. The surgeon can then mark thigh incisions for passage of the needle device 30. Next, the helical needle is passed through the medial thigh incision to meet the surgeon's finger next to the rectum. The arm 42 is fed through the respective perianal incision and the end 48 of the arm 42 is then attached to the distal tip 36 of the needle 30 and the extension portion 46 is pulled through the obturator foramen. The above steps are repeated on the contralateral side, with the other of the two arms 42. The needles are then cut off or otherwise disengaged from the arms, and the base portions 44 of the arms 42 are sutured or otherwise secured to respective sides of the rectum—e.g., one base portion 44 is sutured to a first side of the rectum and another base portion 44 is sutured to a second side of the rectum. The implant 40 is tensioned and the rectum is lifted by pulling on the extension portions 46 at the thigh incisions. For those embodiments including a sheath 49 around the extension portions 46, the sheaths 49 can be removed and the extension portions 46 cut at the thigh incisions. The surgeon can then irrigate the perianal incisions and close the wound in several layers.

### Perineal Approach Rectal Suspension

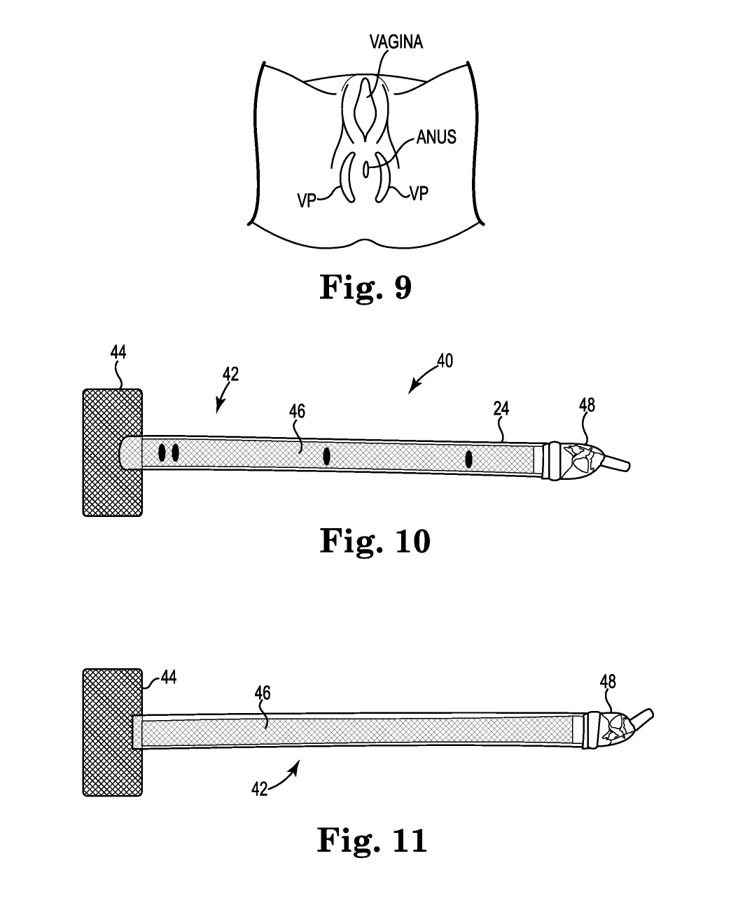

Referring to FIGS. 12-14, embodiments of the present invention can include a perineal approach rectal suspension implant system and method to treat rectal prolapse. The system can include an implant 52 having an attachment portion 54 and two extension arms 56. The arms 56 can extend out at various angles from the attachment portion 54 (generally Y-shaped in certain embodiments), and can include an anchor, dilator, needle or like member or device 58 adapted to engage with an introducer needle. The attachment portion 54 can take on wide variety of shapes and sizes, and the portions 54, 56 can be constructed of a polymer or like mesh material.

With such embodiments, the attachment portion 54 is sutured or otherwise attached to the anterior wall of the rectum, wherein the arms 56 provide rectal suspension at the level of the ischial spine. A perineal incision is provided above the external anal sphincter and dissection is continued along the rectum towards the “Pouch of Douglas” (e.g., rectouterine pouch or “cul-de-sac”). The suspension arms 56 pass through the levator muscle near the ischial spine to traverse the ischiorectal fossa and exit at buttock incisions approximately 3 cm lateral and 3 cm posterior to the anus. The attachment portion 54 can be anchored, sutured or otherwise attached on the rectal wall proximate the Pouch of Douglas. This procedure pulls the rectum back towards the sacrum such that the arms approximate the relevant supporting ligaments.

First, the patient is placed in the lithotomy position, and a perineal incision P (FIG. 12) is created approximately 2 cm below the vaginal introitus. Dissection starts at the external anal sphincter and continues along the rectum towards the Pouch of Douglas. The ischial spine is then palpated. Next, the buttocks is marked approximately 3 cm lateral and 3 cm posterior to the anus and a small stab incision is made at each of the marks. A long curved introducer needle 60 can be passed through the ischiorectal fossa to pierce the levator muscle at the ischial spine. The needle 60 can include a handle 62 and a curved needle 64 having a distal tip portion 66. An arm 56 of the implant 52 can then be connected to the distal tip portion 64 and pulled through the buttocks incision. These steps are then repeated on the contralateral side, connecting and pulling through the other of the arms 56. The attachment portion 54 is laid flat over the rectum and tacked down or otherwise attached to ensure the mesh remains flat after deployment and implantation. The surgeon can pull on the arms 56 to tighten and tension the implant 52 along the rectum.

### Single Incision Perineal Approach Rectal Suspension

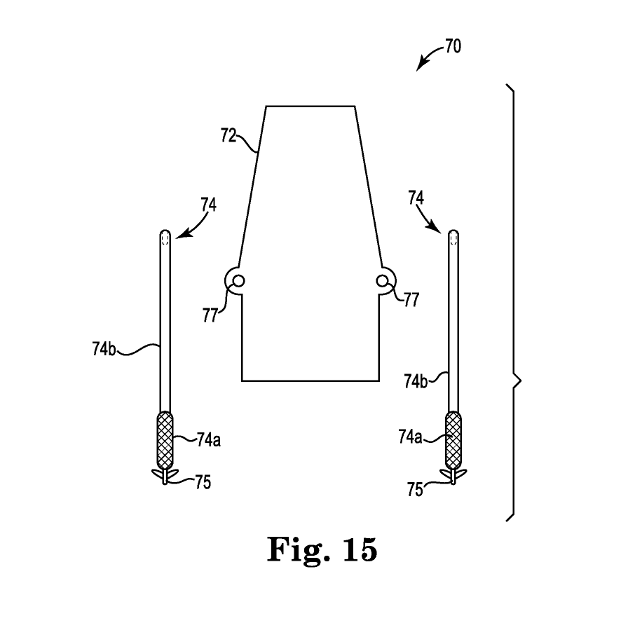

Referring to FIGS. 15-17, the present invention can include a single incision embodiment of the perineal approach rectal suspension implant system and method to treat rectal prolapse. The system can include an implant 70 having a mesh support portion 72 and one or more anchoring arms 74.

The anchoring arms 74 can include a mesh-portion 74a and a non-mesh portion 74b, but entirely mesh extension portion pieces could be used. Distal end portions of the arms 74 can include tissue anchors 75 or like structures or features. An optional adjusting tool 79 can be used to matingly engage the arms 74 with eyelets, grommets or other features 77 provided with the support portion 72. The various tools, devices, implants, and methods disclosed in U.S. Patent Publication Nos. 2010/0261955 and 2010/0274074 can be employed, in full or in part, with the present invention and, therefore, are incorporated fully herein by reference.

Such embodiments are similar to the methods of other embodiments, except the anterior rectal wall is suspended to the sacrospinous ligament using the anchoring arms 74. The perineal approach allows mesh to be placed on the anterior rectal wall similar to ventral rectopexy procedures, avoiding deep mobilization of the rectum posteriolaterally which leads to denervation of the rectum and affects transit time.

First, the patient is placed in the lithotomy position, and a perineal incision P is created approximately 2 cm below the vaginal introitus (e.g., FIG. 12) and dissection is started at the external anal sphincter and continues along the rectum towards the Pouch of Douglas. The surgeon can palpate at the ischial spine and approximately 1.5 cm along the sacrospinous ligament. A deployment needle device 80 includes a handle portion 82, a curved needle 84 and a distal end portion 86. The handle portion 82 can include one or more actuators 88 operatively connected to the end portion 86 to allow the user to selectively engage or disengage a portion of the connected implant 70 (e.g., anchors 75) with the device 80. One of the anchoring arms 74 is connected to the deployment needle device 80, with the needle guided towards the sacrospinous ligament wherein the anchoring arm 74 is deployed or otherwise engaged (e.g., engaging anchor 75) with the ligament approximately 1-2 cm from the ischial spine. These steps are repeated on the contralateral side, engaging the other arm 74 with the sacrospinous ligament. The support portion 72 is then threaded or otherwise connected with both anchoring arms 74 (e.g., via eyelets 77) and pushed into place or securement. The support portion 72 is laid flat over the rectum and tacked down, or otherwise secured, to ensure the mesh remains generally flat to the rectum. The support portion 72 can be further secured in place with the arms 74 with locking eyelets, grommets or other connecting features, devices or mechanism. Any excess mesh or other material can be cut off from the anchoring arms 74, and the wound can be irrigated and closed in several layers.

### Single Incision Posterior Rectal Suspension

Referring again to FIGS. 15-17, embodiments of the present invention can include a system and method of deploying an implant or sling under the rectum to treat rectal prolapse. The implant 70 and its components and structures are deployed via a Kraske incision as described herein, allowing dissection deep along the posterior rectal wall so the mesh will lay flat. The dissection in the ischiorectal fossa is relatively easy without major impeding structures.

The anchors 75 of the arms 74 are secured in the obturator internus muscle leaving the opposing portion of the arms 74 hanging out of the Kraske incision. The support portion 72 can be threaded over, or otherwise connected to the arms 74 and secured up towards the rectum to lay flat. The portion 72 can rest against the puborectalis muscle and the iliococcygeus muscle.

First, the patient can be placed in the lithotomy position. Next, a 3 cm Kraske incision can be performed (e.g., approximately 2 cm inferior to the anus). A Lone Star or like retractor 30 can be used to improve visualization and facilitate introduction of the implant 70. A blunt dissection is made to approximately 1-2 cm beyond the anorectal junction (e.g., transition from the rectum to the anal canal), and the wound is irrigated. One of the anchoring arms 74 is then secured to a delivery needle device and guided to the obturator internus muscle. The tissue anchor 75 is deployed into the obturator internus muscle. The same steps are repeated on the other side, using the other arm 74 of the implant 70. The support portion 72 is then secured in place with the arms 74 with locking eyelets, grommets or other connecting features, devices or mechanism, and appropriately tensioned such that the support portion 72 lays flat against the posterior rectal wall. Any excess mesh or other material can be cut off from the anchoring arms 74, and the wound can be irrigated and closed in several layers.

### Perineal Single Incision Rectopexy

Each of the previously-detailed embodiments suspend and anchor the rectum to anterior anchor points, e.g., the obturator muscle, the sacrospinous ligament or at the ischial spine. The instant embodiment comprises either a perineal incision or a Kraske incision. Dissection can extend along the rectum approximately 3-4 cm and then move to the right lateral rectal wall (physician's left side). A tunneling device can be used to proceed through the tissue under direct visualization to the sacral promontory. Once at the promontory, a strip of mesh can be anchored to the periosteum and the mesh of the implant can be sutured along the left lateral rectal wall.

The implants of the various embodiments disclosed herein can be of any suitable size and shape. For the embodiments where the implant can lay inferior to the rectum, a mesh body of approximately 6×6 cm or 5×6 cm can be used. Mesh arms can be generally 1.1 cm. For embodiments where mesh is placed on the anterior rectal wall, the implants may be 2 cm in width and any suitable length. The implants can be any suitable material, including knitted, polypropylene monofilament, unitary construct (e.g., molded, etched or extruded), or a combination of materials or configurations.

The invention can also include locking connectors disposed at the end of the insertion sheaths. The connectors can be configured to attach to stainless steel curved, helical or straight needles. Insertion sheaths can cover the mesh to facilitate insertion and positioning in the pelvic cavity.

Mesh implant arms can be covered with insertion the sheaths 27. Various shaped needles can be employed as needed depending on the surgical placement of the mesh device.

The invention described herein is envisioned for use with many known implant and repair systems (e.g., for male and female), features and methods, including those disclosed in U.S. Pat. Nos. 8,211,005, 8,206,281, 7,500,945, 7,407,480, 7,351,197, 7,347,812, 7,303,525, 7,025,063, 6,691,711, 6,648,921, and 6,612,977, International Patent Publication Nos. WO 2008/057261 and WO 2007/097994, and U.S. Patent Publication Nos. 2010/0105979, 2002/151762 and 2002/147382. Accordingly, the above-identified disclosures are fully incorporated herein by reference in their entirety.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

What we claim is:

1. A method of treating rectal prolapse, comprising:
providing an implant including a central support portion and first and second extending arms;
providing a needle introducer device having a distal end portion;
creating a transverse incision below the anus of a patient;
attaching the first arm to the distal end portion of the needle introducer;
passing a portion of the first arm through the obturator foramen;
attaching the second arm to the distal end portion of the needle introducer;
passing a portion of the second arm through the obturator foramen; and
positioning the central support portion of the implant generally flat against the rectum.

2. The method of claim 1, wherein the first and second arms further include distal anchors to pass through the obturator foramen.

3. The method of claim 1, wherein the needle introducer includes a generally helical needle portion.

4. The method of claim 1, wherein the central support portion is constructed of a mesh material.

5. The method of claim 1, wherein the first and second arms include distal portions to engage with the distal end portion of the needle device.

6. The method of claim 5, wherein passing a portion of the first arm through the obturator foramen further includes pulling the distal portion of the first arm through a thigh incision.

7. The method of claim 1, wherein the transverse incision below the anus is a Kraske incision.

8. A method of treating rectal prolapse, comprising:
providing an implant including first and second generally T-shaped arms, each T-shaped arm having a base portion and an extension portion, wherein the extension portion extends generally transverse to the base portion.
creating bilateral vertical perianal incisions in a patient;
passing a portion of the extension portion of the first arm through the obturator foramen;
attaching the base portion of the first arm to a first side of the rectum;
passing a portion of the extension portion of the second arm through the obturator foramen; and
attaching the base portion of the second arm to a second side of the rectum.

9. The method of claim 8, further including providing a helical introducer needle device to connect to and deploy the first and second generally T-shaped arms.

10. The method of claim 8, wherein the base portion and the extension portion of the first and second arms are constructed of a mesh material.

11. A system for treating rectal prolapse, comprising:
an implant having a support portion and first and second anchoring arms, the first and second anchoring arms including distal tissue anchors engageable with the sacrospinous ligament of a patient via a perineal incision such that the support portion engages with and stabilizes the rectum.

12. The system of claim 11, further including a generally curved introducer needle device.

13. The system of claim 11, wherein support portion further includes one or more eyelets, and the first and second anchoring arms are separate from the support portion, with at least one of the first and second arms being engageable with the one or more eyelets.

14. The system of claim 13, wherein the first and second arms each include non-mesh portion and a mesh portion.

15. The system of claim 14, wherein the non-mesh portion is a cylindrical rod member.
